(19) United States
(12) Patent Application Publication
(10) Pub. No.: US 2008/0194916 A1
(43) Pub. Date: Aug. 14, 2008

(54) Method, Measurement Device, Computer Program and System for Providing a User with Feedback an Activity

(76) Inventors: Ilkka Limma, Kempele (FI); Ville Kampman, Oulu (FI)

Correspondence Address: NATH & ASSOCIATES, 112 South West Street, Alexandria, VA, 22314, US

(21) Appl. No.: 10/581,315

(22) PCT Filed: Dec. 7, 2004
(86) PCT No.: PCT/FI04/00742

§ 371 Date: Jun. 2, 2006

(30) Foreign Application Priority Data
Dec. 5, 2003 (FI) 20031778

(51) Int. Cl. A61B 5/00

(52) U.S. Cl. 600/300; 701/213

(57) ABSTRACT

The invention discloses a method, measurement device, computer program and system for tranmitting and comparing a recorded activity to an ongoing activity. In the method previously recorded measurement data relating to an activity is loaded into a memory of the measurement device. The loaded recorded measurement data in the memory is compared, during an ongoing activity, to current measurement data measured with the measurement device; and a user of the measurement device is provided with a feedback in response to the comparison.

### FIELD OF THE INVENTION

The present invention relates partly to position determining. In particular, the present invention relates to a novel and improved method, measurement device, computer program and system for comparing a recorded activity to an ongoing activity.

### BACKGROUND OF THE INVENTION

People have always been interested in how they perform in various tasks, e.g. in various sport events. Different manufacturers have provided various kinds of devices that can be used to analyze e.g. a sport event. These devices include e.g. a heart rate monitor, a wrist computer etc.

Global Positioning System (GPS) provides a service in which by using a special GPS receiver position information can be acquired. The GPS uses a plurality of satellites to determine the position.

There exists a plurality of known solutions that use the GPS to log a path e.g. of a sport event. Such a data logger can be afterwards connected to a computer and the performed path, e.g. a run path, can be displayed on a screen of the computer by connecting the recorded position points (coordinates). Furthermore, there exist known solutions that are able to log e.g. heart rate during an exercise. Such a recorded measurement typically provides a user with a great amount of data relating e.g. to an exercise.

When a user uses a measurement device during an activity, it would be desirable to provide the user with some feedback relating to the activity. From prior art is known several solutions with which the user can be shown the current heart rate, speed, distance, altitude etc. The aforementioned quantities tell the user how he/she is performing at that particular moment. Furthremore, it would desirable that the user could be provided with feedback that would tell the user how he/she is performing e.g. compared to a previous occasion.

One known solution trying to solve the aforementioned need uses beforehand set limits for an activity. Based on the set limits, the user can be provided with feedback. For example, it may be set in the measurement device a desired average speed, e.g. 15 km/h) the user should be able to maintain. If the user falls below the limit, the device may give some feedback (e.g. a sound signal, a flashing display etc.). In one prior art solution, based on the set average speed and overall length of the route, it is also possible to calculate during an activity how much the user deviates from the set values. For example, in some point the user may be 25 seconds behind the set, desired exercise level.

An obvious drawback of the aforementioned solutions is that a static target value, e.g. an average speed value, is set to an exercise. The prior art solutions do not take into account e.g. the altitude profile of a particular route. It is evident that it is impossible to keep up a desired speed when climbing e.g. a sharp rise.

Based on the above there is an obvious need for a solution that would enable a user to have more accurate feedback from a measurement device during an activity or exercise.

### SUMMARY OF THE INVENTION

According to one aspect of the invention, there is provided a method of comparing a previously recorded activity to an ongoing activity using a measurement device. The method comprises the step of loading previously recorded measurement data relating to an activity into a memory of the measurement device; comparing during an activity the loaded recorded measurement data in the memory to current measurement data measured with the measurement device; and providing a user of the measurement device with a feedback in response to the comparison.

In one embodiment of the invention, the measurement data comprises at least a plurality of consecutive measurement point sets, each set including at least one heart rate measurement, a time stamp, a GPS position measurement and an altitude measurement.

In one embodiment of the invention, the step of comparing comprising comparing corresponding measurement points of the recorded measurement data in the memory and the current measurement data measured with the measurement device.

In one embodiment of the invention, the step of comparing further comprising comparing at least one of an elapsed time, speed, distance and heart rate.

In one embodiment of the invention, the method further comprises the step of setting at least one predetermined limit for providing the user of the measurement device with feedback.

In one embodiment of the invention, the method further comprises the step of providing the user of the measurement device with feedback only when the at least one predetermined limit is exceeded.

In one embodiment of the invention, the method further comprises the step of providing the user of the measurement device with feedback only when the at least one predetermined limit is gone under.

In one embodiment of the invention, the step of providing comprises providing the user with feedback comprising sound signals.

In one embodiment of the invention, the step of providing comprises providing the user with feedback comprising visually readable feedback from a display.

In one embodiment of the invention, the display is integrated to the measurement device.

In one embodiment of the invention, the display is an external device connected to the measurement device.

According to another aspect of the invention, there is provided a measurement device for recording an activity and comparing a recorded activity to an ongoing activity. The measurement device comprises a data processing unit; a memory connected to the data processing unit; input means configured to receive recorded measurement data relating to an activity, wherein the recorded measurement data is stored on the memory; feedback means configured to provide feedback to the user of the measurement device, wherein the data processing unit is configured to compare during an activity the loaded recorded measurement data in the memory to current measurement data measured with the measurement device and wherein feedback means are configured to provide the user of the measurement device with a feedback in response to the comparison.

In one embodiment of the invention, the measurement data comprises at least a plurality of consecutive measurement point sets, each set including at least one heart rate measurement, a time stamp, a GPS position measurement and an altitude measurement.

In one embodiment of the invention, the data processing unit is configured to compare corresponding measurement points of the recorded measurement data in the memory and the current measurement data measured with the measurement device.

In one embodiment of the invention, the data processing unit is further configured to compare at least one of an elapsed time, speed, distance and heart rate.

In one embodiment of the invention, the data processing unit is configured to set at least one predetermined limit for providing the user of the measurement device with feedback.

In one embodiment of the invention, feedback means are configured to provide the user of the measurement device with feedback only when the at least one predetermined limit is exceeded.

In one embodiment of the invention, feedback means are configured to provide the user of the measurement device with feedback only when the at least one predetermined limit is gone under.

In one embodiment of the invention, feedback means are configured to provide the user with feedback using sound signals.

In one embodiment of the invention, feedback means are configured to provide the user with feedback using visually readable feedback from a display.

In one embodiment of the invention, the display is integrated to the measurement device.

In one embodiment of the invention, the display is an external device connected to the measurement device.

In one embodiment of the invention, the measurement device is a hand-held measurement device.

According to another aspect of the invention, there is provided a computer program for comparing a previously recorded activity to an ongoing activity, the computer program comprising code adapted to perform the following steps when executed on a data-processing device: loading previously recorded measurement data relating to an activity into a memory of the measurement device; comparing during an activity the loaded recorded measurement data in the memory to current measurement data measured with the measurement device; and providing a user of the measurement device with a feedback in response to the comparison.

In one embodiment of the invention, the measurement data comprises at least a plurality of consecutive measurement point sets, each set including at least one heart rate measurement, a time stamp, a GPS position measurement and an altitude measurement.

In one embodiment of the invention, the step of comparing comprising comparing corresponding measurement points of the recorded measurement data in the memory and the current measurement data measured with the measurement device.

In one embodiment of the invention, the step of comparing further comprising comparing at least one of an elapsed time, speed, distance and heart rate.

In one embodiment of the invention, the computer program is further adapted to perform the following step when executed on said data-processing device: setting at least one predetermined limit for providing the user of the measurement device with feedback.

In one embodiment of the invention, the computer program is further adapted to perform the following step when executed on said data-processing device: providing the user of the measurement device with feedback only when the at least one predetermined limit is exceeded.

In one embodiment of the invention, the computer program is further adapted to perform the following step when executed on said data-processing device: providing the user of the measurement device with feedback only when the at least one predetermined limit is gone under.

In one embodiment of the invention, the step of providing comprising providing the user with feedback comprising sound signals.

In one embodiment of the invention, the step of providing comprising providing the user with feedback comprising visually readable feedback from a display.

In one embodiment of the invention, the computer program is stored on a computer readable medium.

According to yet another aspect of the invention, there is provided a system for transferring measurement data relating to an activity. The system comprises a computer; a first memory in the computer, the first memory comprising recorded measurement data relating to an activity; a measurement device connected to the computer; a second memory in the measurement device configured to store measurement data; output means configured to output the recorded measurement data relating to an activity; and input means configured to receive previously recorded measurement data relating to an activity via the output means, wherein the previously recorded measurement data is stored on the second memory of the measurement device.

In one embodiment of the invention, the measurement data comprises at least a plurality of consecutive measurement point sets, each set including at least one heart rate measurement, a time stamp, a GPS position measurement and an altitude measurement.

In one embodiment of the invention, the system further comprises setting means configured to set at least one predetermined limit in the second memory of the measurement device in order to provide a user of the measurement device with feedback during the activity.

The present invention has several advantages over the prior-art solutions. Thanks to the invention a user is able accurately to compare his/her current performance to measurement data stored before the activity to a measurement device. The measurement device continuously measures the ongoing activity, and at the same time, provides the user with feedback, e.g. via a display device or sound signals.

### BRIEF DESCRIPTION OF THE DRAWINGS

The accompanying drawings, which are included to provide a further understanding of the invention and constitute a part of this specification, illustrate embodiments of the invention and together with the description help to explain the principles of the invention. In the drawings:

FIG. 1 illustrates one embodiment of a method according to the present invention;

FIG. 2 illustrates one embodiment of a measurement device according to the present invention, and

FIG. 3 illustrates one embodiment of a system according to the present invention.

### DETAILED DESCRIPTION OF THE INVENTION

Reference will now be made in detail to the embodiments of the present invention, examples of which are illustrated in the accompanying drawings.

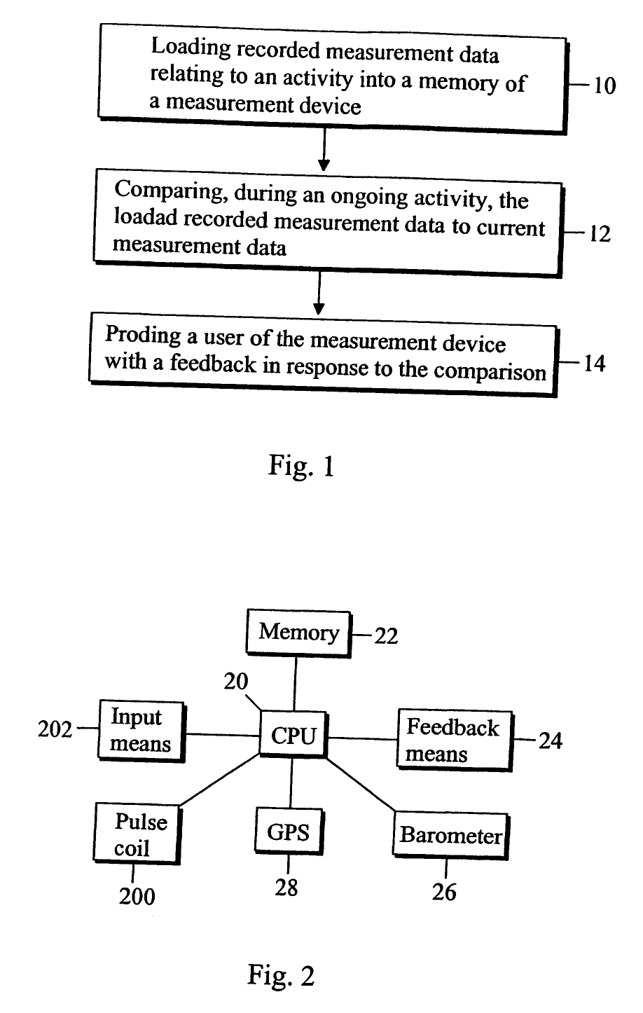

FIG. 1 discloses one embodiment of a method according to the invention.

At step 10 previously recorded measurement data relating to an activity is loaded into a memory of a measurement device. The measurement data comprises a plurality of consecutive measurement point sets, each set including e.g. a heart rate measurement, a time stamp, a GPS position measurement and an altitude measurement. The measurement data may have been recorded by a user earlier using his/her own measurement device. After the activity, the measurement data was loaded into a computer for further analysis. The measurement data may alternatively refer to data that someone else has recorded using his/her own measurement device. The measurement data files can be easily transferred between different computers. In another embodiment, the measurement data is an old measurement of the user already in the memory of the measurement device.

When the user starts a new measurement data recording, new data is compared with the measurement data in the memory of the measurement device during the ongoing activity, as indicated at step 12. In one embodiment, the loaded measurement data and measurement data of the ongoing activity are directly comparable. In other words, measurements in the loaded file can directly be compared to measurements measured with the measurement device. In another embodiment, the loaded measurement data comprises derived information based on actual actual measurement data. In other words, now measurement data of the ongoing activity cannot be directly compared with the loaded measurement data. Therefore, a processor of the measurement device may have to calculate one or more values based measurement data of the ongoing activity, and then compare the results with the loaded measurement data. Yet in another embodiment, a combination of the two previous embodiments is used. The compared values include e.g. an elapsed time, speed, distance and heart rate.

In order to be able to compare previously recorded measurement data in the memory of the measurement device and measurement data relating to an ongoing activity, both measurements are preferably recorded essentially in the same type of route or path.

After the comparison, the user can be provided with a feedback, as indicated at step 14. The feedback may include sound signals, visually readable feedback or any other appropriate form of feedback. The feedback may be e.g. that the user is shown his/her current speed and, at the same time, the speed value at the same point in the loaded measurement data. The same technique can be applied e.g. to time (used time so far versus used time in the loaded measurement), distance (how far the user has been able to get versus the situation in the loaded measurement) In another embodiment, the user may be shown on a display of the measurement device or on a display device connected to the measurement device how much he/she as fallen below (in second, minutes, etc.) compared with the virtual friend (the loaded measurement data).

In one embodiment, when the recorded measurement data is loaded into the memory of the measurement device, also some predetermined limits are set for providing the user with feedback. The user may have e.g. set that he/she has to perform 3% better compared with the loaded measurement data. Therefore, the user may be provided with feedback (sound signals, flashing display etc.) e.g. he/she falls below the 3% improvement limit.

FIG. 2 discloses one embodiment of a measurement device according to the invention. It must be noted that the measurement device includes also other elements and components not shown in FIG. 2.

The measurement device of FIG. 2 includes a central processing unit (CPU) 20 containing the logic circuitry that performs the instructions of needed programs. The device further includes a memory 22 into which measurement data can be loaded and into which measurement data of an ongoing activity is stored. Memory 20 may be a single memory or it may consist of several individual memories. Memory 20 may also comprise software needed to implement the functionality disclosed in the invention.

The measurement device is also provided with several elements that produce different quantities to be stored on memory 22:
a GPS receiver 28 for receiving position information from a plurality of satellites.
The satellites further provide needed time stamps stating the current time.
A barometer 26 for measuring air pressure.
A pulse coil 200 for receiving heart rate information from a heart rate belt.

It is evident that in other embodiments of FIG. 2 the measurement device may contain also additional elements not shown in FIG. 2 measuring other value/quantities. In other embodiments, any one of pulse coil 200, GPS 28 and barometer 26 or a combination of them may be left out from the measurement device.

The measurement device further includes input means 202 configured to receive recorded measurement data relating to a previous activity e.g. from a computer connected to the measurement device. The data received measurement data received with input means 200 is stored on memory 22. Feedback means 24 are provided to give feedback to a user of the measurement device during an ongoing activity. The feedback issue was discussed in more detail with FIG. 2.

CPU 20 is configured to compare corresponding measurement points of the recorded measurement data in memory 22 and the current measurement data measured with the measurement device. CPU 22 may further be configured to compare at least one of an elapsed time, speed, distance and heart rate. In one embodiment, the loaded measurement data in memory 22 comprises derived information based on actual actual measurement data. In other words, now measurement data of the ongoing activity cannot be directly compared with the loaded measurement data. Therefore, CPU 20 is configured to calculate one or more values based measurement data of the ongoing activity, and then compare the results with the loaded measurement data. Yet in another embodiment, a combination of the two previous embodiments is used.

CPU 20 may also be configured to set at least one predetermined limit for providing the user of the measurement device with feedback during an ongoing activity. This was also discussed in more detail with FIG. 2. Feedback means 24 provide the user with e.g. sound signals, readable feedback etc. Based on the above, it is evident that feedback means 24 may actually refer to more than one device, component or element providing the actual feedback, e.g. to a loudspeaker, display, vibrator etc. In one embodiment, the display is integrated to the measurement device. In another embodiment, the display is an external device connected to the measurement device.

The measurement device is preferably a hand-held device that can easily be fastened e.g. to an arm or a wrist.

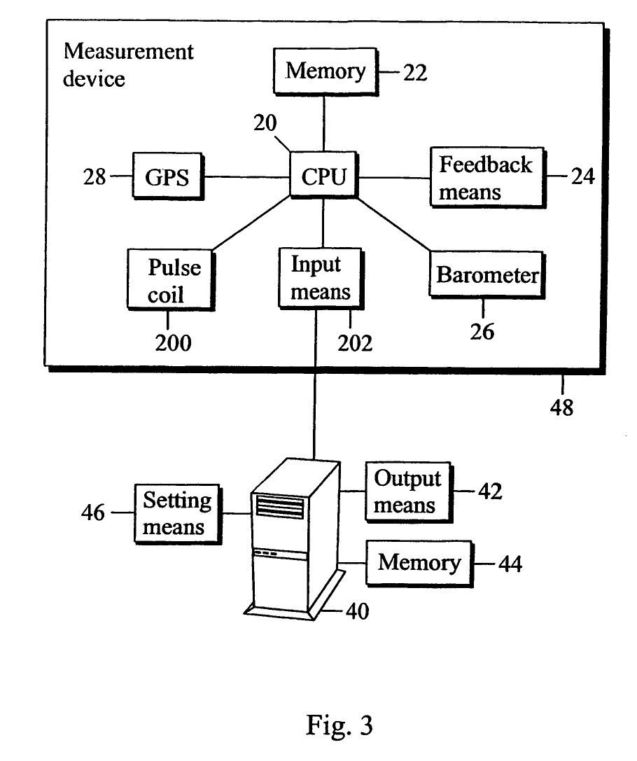

FIG. 3 discloses one embodiment of a system according to the invention. The system comprises a similar measurement device 48 as disclosed in FIG. 2. Therefore, measurement device 48 is not discussed in more detail.

The system comprises also a computer 40 and a memory 44 in computer 40. Memory 44 comprises previously recorded measurement data relating to an activity. It is evident that computer 40 includes also other components, such as a display, a CPU etc not shown in FIG. 3. FIG. 3 discloses only elements that are needed to explain the system according to the invention.

Computer 40 includes also output means 42 configured to output recorded measurement data relating to an activity to measurement device 48. Input means 202 of measurement device 48 are configured to receive the recorded measurement data. The measurement data comprises at least a plurality of consecutive measurement point sets, each set including e.g. a one heart rate measurement, a time stamp, a GPS position measurement and an altitude measurement.

Computer 40 includes also setting means 46 configured to set at least one predetermined limit in memory 22 of measurement device 48. The at least one limit may be used when feedback means 24 of measurement device 48 provide user with feedback during an ongoing activity. Functionality relating to the at least one predetermined limit was discussed in more detail with FIG. 2.

Output means 42, setting means 46 and input means 202 are implemented in a way known to a man skilled in the art, e.g. using hardware and software components and elements, and therefore they are not described in detail.

It is obvious to a person skilled in the art that with the advancement of technology, the basic idea of the invention may be implemented in various ways. The invention and its embodiments are thus not limited to the examples described above, instead they may vary within the scope of the claims.

1. A method of comparing a previously recorded activity to an ongoing activity using a measurement device,
characterised in that the method comprises:
loading measurement data of a previously recorded real continuous activity recorded on a route into a memory of the measurement device;
comparing, during an ongoing continuous activity, the loaded recorded measurement data in the memory to current measurement data measured with the measurement device on essentially the same type of route as the previously recorded real continuous activity; and
providing a user of the measurement device With a feedback in response to the comparison at any given time.

2. The method according to claim 1, characterised in that the measurement data comprises at least a plurality of consecutive measurement point sets, each set including at least one heart rate measurement, a time stamp, a GPS position measurement and an altitude measurement.

3. The method according to claim 1, characterised in that said step of comparing comprising comparing corresponding measurement points of the previously recorded measurement data in the memory and the current measurement data measured with the measurement device.

4. The method according to claim 3, characterised in that said step of comparing further comprising comparing at least one of an elapsed time, speed, distance and heart rate.

5. The method according to claim 1, characterised in that the method further comprises the step of:
setting at least one predetermined limit for providing the user of the measurement device with feedback.

6. The method according to claim 5, characterised in that the method further comprises the step of:
providing the user of the measurement device with feedback only when the at least one predetermined limit is exceeded.

7. The method according to claim 5, characterized in that the method further comprises the step of:
providing the user of the measurement device with feedback only when the at least one predetermined 15 limit is gone under.

8. The method according to claim 1, characterised in that said step of providing comprising providing the user with feedback comprising sound signals.

9. The method according to claim 1, characterised in that said step of providing comprising providing the user with feedback comprising visually readable feedback from a display.

10. The method according to claim 9, characterised in that the display is integrated to the measurement device.

11. The method according to claim 9, characterised in that the display is an external device connected to the measurement device.

12. A measurement device for recording an activity and comparing a recorded activity to an ongoing activity,
characterised in that the measurement device comprises:
a data processing unit (20);
a memory (22) connected to the data processing unit (20);
input means (202) configured to receive measurement data of a previously recorded real continuous activity recorded on a route, wherein the recorded measurement data is stored on the memory (22);
feedback means (24) configured to provide feedback to the user of the measurement device;
wherein the data processing unit (20) is configured to compare during an ongoing continuous activity the loaded recorded measurement data in the memory (22) to current measurement data measured with the measurement device on essentially the same type of route as the previously recorded real continuous activity and wherein feedback means (24) are configured to provide the user of the measurement device with a 15 feedback in response to the comparison at any given time.

13. The measurement device according to claim 12, characterised in that the measurement data comprises at least a plurality of consecutive measurement point sets, each set including at least one heart rate measurement, a time stamp, a GPS position measurement and an altitude measurement.

14. The measurement device according to claim 12, characterised in that the data processing unit (20) is configured to compare corresponding measurement points of the recorded measurement data in the memory (22) and the current measurement data measured with the measurement device.

15. The measurement device according to claim 14, characterised in that the data processing unit (20) is further configured to compare at least one of an elapsed time, speed, distance and heart rate.

16. The measurement device according to claim 12, characterised in that the data processing unit (20) is configured to set at least one predetermined limit for providing the user of the measurement device with feedback.

17. The measurement device according to claim 16, characterised in that the feedback means (24) are configured to provide the user of the measurement device with feedback only when the at least one predetermined limit is exceeded.

18. The measurement device according to claim 16, characterised in that the feedback means (24) are configured to provide the user of the measurement device with feedback only when the at least one predetermined limit is gone under.

19. The measurement device according to claim 12, characterised in that the feedback means (24) are configured to provide the user with feedback using sound signals.

20. The measurement device according to claim 12, characterised in that the feedback means (24) are configured to provide the user with feedback using readable feedback from a display.

21. The measurement device according to claim 20, characterised in that the display is integrated to the measurement device.

22. The measurement device according to claim 20, characterised in that the display is an external device connected to the measurement device.

23. The measurement device according to claim 12, characterized in that the measurement device is a hand-held measurement device.

24. A computer program for comparing a previously recorded activity to an ongoing activity, the computer program comprising code adapted to perform the following steps when executed on a data-processing device:
loading measurement data of a previously recorded real continuous activity on a route into a memory of a measurement device;
comparing, during an ongoing continuous activity, the loaded recorded measurement data in the memory to current measurement data measured with the measurement device on essentially the same type of route as the previously recorded real continuous activity; and
providing a user of the measurement device with a feedback in response to the comparison at any given Lime.

25. The computer program according to claim 24, characterised in that the measurement data comprises at Least a plurality of consecutive measurement point sets, each set including at least one heart rate measurement, a time stamp, a GPS position measurement and an altitude measurement.

26. The computer program according to claim 24, characterised in that said step of comparing comprising comparing corresponding measurement points of the previously recorded measurement data in the memory and the current measurement data measured with the measurement device.

27. The computer program according to claim 26, characterised in that said step of comparing further comprising comparing at least one of an elapsed time, speed, distance and heart rate.

28. The computer program according to claim 24, characterised in that the computer program is further adapted to perform the following step when executed on said data-processing device:
setting at least one predetermined limit for providing the user of the measurement device with feedback.

29. The computer program according to claim 28, characterised in that the computer program is further adapted to perform the following step when executed on said data-processing device:
providing the user of the measurement device with feedback only when the at least one predetermined limit is exceeded.

30. The computer program according to claim 28, characterised in that the computer program is further adapted to perform the following step when executed on said data-processing device:
providing the user of the measurement device with feedback only when the at least one predetermined limit is gone under.

31. The computer program according to claim 24, characterised in that said step of providing comprising providing the user with feedback comprising sound signals.

32. The computer program according to claim 24, characterised in that said step of providing comprising providing the user with feedback comprising visually readable feedback from a display.

33. The computer program according to claim 24, characterised in that the computer program is stored on a computer readable medium.

34. A system for transferring measurement data relating to an activity, characterised in that the system comprises:
a computer (40);
a first memory (44) in the computer (40), the first memory (44) comprising measurement data of a previously recorded real continuous activity recorded on a route;
a measurement device (48) connected to the computer (40);
a second memory (22) in the measurement device (48) configured to store measurement data on essentially the same type of route as the previously recorded real continuous activity;
output means (42) configured to output the previously recorded measurement data relating to an activity; and
input means (202) configured to receive the measurement data of the previously recorded real continuous activity recorded via the output means (42), wherein the recorded measurement data is stored on the second memory (22) of the measurement device (48).

35. The system according to claim 34, characterised in that the measurement data comprises at least a plurality of consecutive measurement point sets, each set including at least one heart rate measurement, a time stamp, a GPS position measurement and an altitude measurement.

36. The system according to claim 34, characterised in that the system further comprises setting means (46) configured to set at least one predetermined limit in the second memory (22) of the measurement device (48) in order to provide a user of the measurement device (48) with feedback during the activity.
